(19) United States
(12) Patent Application Publication
(10) Pub. No.: US 2014/0275122 A1
(43) Pub. Date: Sep. 18, 2014

(54) Voriconazole Formulations

(71) Applicant: Fresenius Kabi USA, LLC, Lake Zurich, IL (US)

(72) Inventors: Beena Uchil, Lake Forest, IL (US); Joel Hoekstra, Lisle, IL (US)

(73) Assignee: Fresenius Kabi USA, LLC, Lake Zurich, IL (US)

(21) Appl. No.: 14/209,353

(22) Filed: Mar. 13, 2014

Related U.S. Application Data
(60) Provisional application No. 61/783,561, filed on Mar. 14, 2013.

(51) Int. Cl. A61K 31/506; A61K 47/18; A61K 47/26; A61K 9/19; A61K 47/40

(52) U.S. Cl. CPC A61K 31/506; A61K 9/19; A61K 47/40; A61K 47/26; A61K 47/183
USPC 514/256

(57) ABSTRACT

A voriconazole composition includes voriconazole, hydroxypropyl β-cyclodextrin, and an excipient selected from the group consisting of an amino acid and a disaccharide, where the composition is a solid. The solid composition may be made by forming a liquid mixture including a solvent, voriconazole, HPCD, and an excipient selected from the group consisting of an amino acid and a disaccharide, and lyophilizing the liquid mixture.

### CROSS-REFERENCE TO RELATED APPLICATION

This application claims the benefit of U.S. Provisional Patent Application No. 61/783,561, filed Mar. 14, 2013, which is incorporated herein by reference in its entirety.

### BACKGROUND OF THE INVENTION

A variety of fungal infections can occur in patients due to pathogenic Candida, Aspergillus, Fusarium or Scedosporium fungus species. Examples of such fungal infections include candidemia, candidiasis, invasive aspergillosis, scedosporiosis and fusariosis. Historically, these infections have been addressed with polyene antifungal agents such as amphotericin B, or with triazole antifungal agents such as itraconazole and fluconazole. These antifungal agents had a variety of drawbacks, however, including toxic side effects, drug-drug interactions, variations in efficacy between patients, and fungal resistance.

Voriconazole is a more recent triazole antifungal agent that is less prone to the drawbacks of previous antifungal agents. Triazole antifungal agents are believed to treat infections by inhibiting the enzyme 14-α-sterol demethylase, which converts lanosterol to ergosterol as an important step of building fungal cell membranes. Voriconazole is a derivative of the triazole antifungal agent fluconazole. Relative to fluconazole, voriconazole has a broader spectrum of antifungal effectiveness, as voriconazole is believed to inhibit 14-α-sterol demethylase more effectively, including in strains of C. albicans that have developed resistance to fluconazole.

Voriconazole has been approved in the U.S. for treatment of a variety of fungal infections. The full name for voriconazole is (2R,3S)-2-(2,4-difluorophenyl)-3-(5-fluoro-4-pyrimidinyl)-1-(1H-1,2,4-triazol-1-yl)-2-butanol, and a representative chemical structure is shown in FIG. 1. An approved treatment regimen for adults includes administration of two initial doses of 6 milligrams (mg) voriconazole per kilogram (kg) of body weight (mg/kg) every 12 hours for the first 24 hours of treatment, followed by administration of a maintenance dose of 4 mg/kg voriconazole every 12 hours, where each administration is performed through intravenous infusion over 1-2 hours. Voriconazole also may be administered orally, and a patient may switch from intravenous administration to oral tablets or an oral suspension, provided they can tolerate oral administration.

Intravenous administration of voriconazole involves reconstitution of a lyophilized solid containing the voriconazole. In one example, a formulation of voriconazole that is commercially available at present is sold under the VFEND™ trademark. VFEND™ for Injection (Pfizer Inc.; New York, N.Y., USA) is currently available as a lyophilized powder containing 200 mg of voriconazole and 3,200 mg sulfobutyl ether β-cyclodextrin sodium (SBECD). VFEND™ for Injection is reconstituted for administration by combining the lyophilized powder with 19 milliliters (mL) of a reconstitution liquid such as water for injection, to provide a solution having a voriconazole concentration of 10 milligrams per milliliter (mg/mL). An aliquot of 20 mL of this solution, which contains 200 mg voriconazole, preferably is diluted with 20 mL or more of an infusion liquid prior to administration, to provide a solution having a voriconazole concentration of 5 mg/mL or less.

One of the challenges in preparing and using formulations of voriconazole is its insolubility in aqueous liquids. In an aqueous liquid having a pH of 3, the solubility of voriconazole is only 2 mg/mL. Effective aqueous solubilization of voriconazole has been difficult to achieve, as the semi-polarity of voriconazole is believed to inhibit the solubilization effects of conventional solubilizing additives such as oils, surfactants and/or water-miscible solvents. Another challenge in preparing and using formulations of voriconazole is its tendency to degrade into other substances, including an inactive enantiomer, when present in an aqueous liquid over time.

One approach to solubilizing voriconazole has been to combine voriconazole with a sulfonated cyclodextrin. U.S. Pat. No. 6,632,803 discloses formulations of voriconazole with SBECD. Other approaches have included combining voriconazole with hydroxypropyl β-cyclodextrin (HPCD) and glycine in a 1:10 molar ratio of voriconazole to glycine (see EP 2 018 866 and EP 2 409 699); forming microparticles of voriconazole and then dispersing the particles using a surfactant (see WO 2004/032902); encapsulating voriconazole in a liposome (CN 102697726); combining voriconazole with a poly(alkyl ether) (see US 2005/112204) or with a block copolymer of poly(ethylene glycol) and poly(lactic acid) (see US 2011/0257197); using a co-solvent such as N-methyl pyrrolidone (NMP; see EP 2 027 850); and chemically modifying voriconazole with one or more phosphate groups to form a pro-drug of voriconazole (see WO 97/28169).

Although some of these approaches have improved the aqueous solubility stability of voriconazole, lyophilized powders containing voriconazole must still be stored in a controlled environment in order to inhibit degradation of the voriconazole. Current protocols for VFEND™ require the lyophilized powder to be stored at temperatures from 15° C. to 30° C. Moreover, reconstituted liquids containing tigecycline also must be maintained in a controlled environment. Current protocols for VFEND™ allow for reconstituted liquids to be stored at temperatures from 2° C. to 8° C. for 24 hours. Thus, hospital staff presently is burdened with the need to prepare voriconazole mixtures close to the time of administration, and to monitor the temperature and/or administration time of the reconstituted mixtures, all in the context of caring for a critically infected patient.

It is desirable to have voriconazole formulations that can be stored as lyophilized solids without the need for control of the surrounding temperature. For example, it is desirable for a lyophilized formulation of voriconazole to be stable at temperatures above 30° C. for at least 2 years. In another example, it is desirable for a reconstituted formulation of voriconazole to be stable at temperatures above 8° C. for more than 24 hours and/or to be stable at room temperatures (˜25° C.) or above for at least 24 hours. Preferably such stabilized formulations would be convenient to prepare, store, reconstitute and administer.

### BRIEF SUMMARY OF THE INVENTION

A composition is provided that includes voriconazole, HPCD, and an excipient selected from the group consisting of an amino acid and a disaccharide. The composition is a solid.

A composition is provided that includes voriconazole, HPCD, and an excipient selected from the group consisting of arginine, lysine, threonine, lactose and trehalose. The composition is a solid.

A composition is provided that includes voriconazole, HPCD, and arginine. The molar ratio of voriconazole to HPCD is from 1:2.7 to 1:3.5. The molar ratio of voriconazole to arginine is from 1:9 to 1:11. The composition is a solid.

A solid composition is provided, which is formed by a method that includes forming a liquid mixture including a solvent, voriconazole, HPCD, and an excipient selected from the group consisting of an amino acid and a disaccharide. The method further includes lyophilizing the liquid mixture to form a solid composition.

### BRIEF DESCRIPTION OF THE DRAWING

FIG. 1 depicts a chemical structure of voriconazole.

### DETAILED DESCRIPTION OF THE INVENTION

Lyophilized formulations that include voriconazole, HPCD and an amino acid or a disaccharide can protect voriconazole from degradation. These formulations may be stored at room temperature or above for more than 2 years, and thus may not require storage in a refrigerator or freezer prior to use. Reconstitution of the lyophilized formulations with a carrier liquid can yield an injectable liquid that may be used to administer voriconazole. The reconstituted liquid may be stored at room temperature (˜25° C.) for more than 24 hours.

A composition may include voriconazole, HPCD, an amino acid or a disaccharide, and optionally one or more other substances, where the composition is a solid. The solid composition may be prepared by forming a liquid mixture including a solvent, voriconazole, HPCD, and the amino acid or disaccharide, and then lyophilizing the mixture. The resulting solid composition may be used in administering voriconazole to a patient by combining the composition with an aqueous carrier to form a solution or emulsion, which, for example, can be injected into a patient.

A solid composition that includes voriconazole, HPCD, and an amino acid or disaccharide may include an amount of voriconazole that is sufficient for a single initial dose of voriconazole, or an amount sufficient for a maintenance dose of voriconazole. A solid composition that includes voriconazole, HPCD, and an amino acid or disaccharide may include an amount of voriconazole that is sufficient for two or more initial doses of voriconazole, or an amount sufficient for two or more maintenance doses of voriconazole. The amount of voriconazole in the composition may be a different therapeutic amount. For example, the amount of voriconazole in the composition may be an amount sufficient for half of an initial dose, or for half of a maintenance dose.

To provide a clear and more consistent understanding of the specification and claims of this application, the following definitions are provided.

The term “mass ratio” of two substances means the mass of one substance (S1) relative to the mass of the other substance (S2), where both masses have identical units, expressed as S1:S2.

The term “lyophilizing” means removing from a solution or an emulsion one or more substances having the lowest boiling points by freezing the solution or emulsion and applying a vacuum to the frozen mixture.

The term “solid” means a substance that is not a liquid or a gas. A solid substance may have one of a variety of forms, including a monolithic solid, a powder, a gel or a paste.

The term “disaccharide” means a carbohydrate having a stoichiometric formula of Cₙ(H₂O)ₙ₋₁ where n is from 10 to 12, and having a chemical structure that includes two aldose and/or ketose molecules linked through a glycosidic bond. Reference to any saccharide by a single name also includes all forms of that saccharide which may be in equilibrium with the specific saccharide named, in aqueous mixture at room temperature.

In one example, a solid composition that includes voriconazole, HPCD, and an amino acid or disaccharide may include from 50 to 500 milligrams (mg) voriconazole. Preferably the composition includes from 100 to 450 mg voriconazole, or from 150 to 250 mg voriconazole. Presently preferred amounts of voriconazole in the composition include about 200 mg and about 400 mg.

A solid composition that includes voriconazole, HPCD, and an amino acid or disaccharide may include an amount of HPCD sufficient to solubilize the voriconazole. Preferably the amount of HPCD in the composition is at most an amount that will solubilize the voriconazole in a sample of aqueous liquid, such as a volume of aqueous liquid used for reconstitution of the solid composition. In one example, the solid composition includes voriconazole and HPCD in a molar ratio of voriconazole to HPCD of from 1:2 to 1:5.5. Preferably the molar ratio of voriconazole to HPCD is from 1:2.2 to 1:5, from 1:2.5 to 1:4, from 1:2.7 to 1:3.5, or from 1:2.9 to 1:3.1. For a solid composition that includes 200 mg voriconazole, together with HPCD and an amino acid or disaccharide, the amount of HPCD may be from 1 to 4 grams, from 2 to 3.5 grams, or from 2.5 to 3.0 grams.

One of ordinary skill in the art can readily calculate the molar ratio of voriconazole to HPCD in a composition of the invention based upon the molecular weights of voriconazole and HPCD and the mass of voriconazole and HPCD included in the composition. Voriconazole free base has a molecular weight of approximately 349.3 g/mol. HPCD is typically supplied as a composite product composed of several molecular species. The average molecular weight of HPCD depends upon the molar substitution (MS), i.e., the average number of hydroxypropyl groups per anhydroglucose unit. The average molecular weight (MWₐᵥₑ) of HPCD can be estimated using the following formula: MWₐᵥₑ=1135+(7×MS×58.1). In some embodiments, the HPCD included in a composition of the invention has a MS in the range of 0.62-0.66, and a MWₐᵥₑ in the range of approximately 1387 g/mol-1403 g/mol. In certain embodiments, the HPCD included in a composition of the invention has a MWₐᵥₑ of about 1395 g/mol.

Cyclodextrins other than HPCD may be used to solubilize voriconazole in a solid composition containing an amino acid or a disaccharide, and optionally one or more other substances. In some embodiments, a hydroxyalkylated β-cyclodextrin other than HPCD is used in a composition of the invention, such as a hydroxyethyl β-cyclodextrin or a dihydroxypropyl β-cyclodextrin. In other embodiments, the cyclodextrin is selected from the group consisting of a branched β-cyclodextrin, a methylated β-cyclodextrin, an ethylated β-cyclodextrin, and an anionic β-cyclodextrin.

A solid composition that includes voriconazole, HPCD, and an amino acid preferably includes arginine, lysine and/or threonine as the amino acid. A solid composition that includes voriconazole, HPCD, and a disaccharide preferably includes lactose and/or trehalose as the disaccharide.

A solid composition that includes voriconazole, HPCD, and an amino acid or disaccharide may include an amount of the amino acid or disaccharide sufficient to stabilize the voriconazole against degradation. Preferably the amount of amino acid or disaccharide in the composition is at most an amount that will stabilize voriconazole against degradation in an aqueous liquid, such as a volume of aqueous liquid used for reconstitution of the solid composition. In one example, the solid composition includes voriconazole and an amino acid or disaccharide in a molar ratio of from 1:2.5 to 1:20, from 1:5 to 1:15, or from 1:9 to 1:11. In another example, the solid composition includes voriconazole and an amino acid or disaccharide in a molar ratio of 1:10. For a solid composition that includes 200 mg voriconazole, together with HPCD and an amino acid or disaccharide, the amount of the amino acid or disaccharide may be from 250 mg to 2 g, from 500 mg to 1.5 g, or from 900 mg to 1 g.

A solid composition that includes voriconazole, HPCD, and an amino acid or disaccharide may further include a pH modifier, such as an acid, a base or a buffer. Examples of acids include hydrochloric acid, acetic acid and citric acid. Examples of bases include sodium hydroxide and ammonium hydroxide. Examples of buffers include citrate buffer, phosphate buffer and tri maleate buffer.

The amount of the pH modifier may be an amount sufficient to provide a pH in the range of from 5 to 7 when a composition containing 200 mg voriconazole is reconstituted in 19 mL of water for injection. Preferably the amount of the pH modifier is an amount sufficient to provide a pH in the range of from 5.2 to 6.9, from 5.5 to 6.7, or from 6.0 to 6.5 when a composition containing 200 mg voriconazole is reconstituted in 19 mL of water for injection.

A solid composition that includes voriconazole, HPCD, and an amino acid or disaccharide may further include one or more other substances. Non-limiting examples of other substances include bulking agents, carriers, diluents, fillers, salts, stabilizers, solubilizers, preservatives, antioxidants, and tonicity contributors. Substances that may be useful in formulating pharmaceutically acceptable compositions, and methods of forming such compositions, are described for example in Remington: The Science and Practice of Pharmacy, 20th Ed., ed. A. Gennaro, Lippincott Williams & Wilkins, 2000, and in Kibbe, “Handbook of Pharmaceutical Excipients,” 3^{rd} Edition, 2000.

Surprisingly, it has been discovered that voriconazole in a solid composition including HPCD and an amino acid or disaccharide may be more stable than voriconazole in a solid composition including SBECD (i.e., VFEND™; Pfizer, Inc.). It is presently believed that solid compositions that include HPCD and an amino acid or disaccharide may be able to protect voriconazole from degradation for more than 2 years at room temperature (˜25° C.), and for at least 2 years at elevated temperatures above room temperature.

It is presently believed that solid compositions that include voriconazole, HPCD, and an amino acid or disaccharide may be able to protect voriconazole from degradation for more than 2 years at room temperature (˜25° C.), and for at least 2 years at elevated temperatures above 25° C. Preferably, when a solid composition including voriconazole, HPCD, and an amino acid or disaccharide is stored at 25° C. over a period of 12 months, at most 1% of the voriconazole degrades. Degradation of voriconazole includes any conversion of voriconazole into a different substance, including but not limited to an enantiomer or a related compound. Methods for identifying and quantifying voriconazole degradation products are well-known to those of skill in the art. In certain embodiments, voriconazole degradation products are assessed by high-performance liquid chromatography (HPLC).

Preferably, when a solid composition including voriconazole, HPCD, and an amino acid or disaccharide is stored at 40° C. over a period of 4 weeks, at most 0.65% of the voriconazole degrades. More preferably, when a solid composition including voriconazole, HPCD, and an amino acid or disaccharide is stored at 40° C. over a period of 4 weeks, at most 0.6%, 0.55%, 0.5%, 0.45%, 0.4%, 0.35%, 0.3%, 0.25% or 0.20% of the voriconazole degrades.

Preferably, when a solid composition including voriconazole, HPCD, and an amino acid or disaccharide is stored at 55° C. over a period of 2 weeks, at most 2.2% of the voriconazole degrades. More preferably, when a solid composition including voriconazole, HPCD, and an amino acid or disaccharide is stored at 55° C. over a period of 2 weeks, at most 2%, 1.7%, 1.5%, 1.0%, 0.9%, 0.8%, 0.7%, 0.6%, 0.5%, 0.4% or 0.3% of the voriconazole degrades.

It is presently believed that reconstituted liquids formed from solid compositions including voriconazole, HPCD, and an amino acid or disaccharide may be able to protect voriconazole from degradation in solution form for more than 24 hours at 25° C., and for at least 24 hours at elevated temperatures above 25° C. Preferably, when a solid composition including voriconazole, HPCD, and an amino acid or disaccharide is reconstituted and stored at 25° C. over a period of 24 hours, at most 0.5% of the voriconazole degrades. More preferably, when a solid composition including voriconazole, HPCD, and an amino acid or disaccharide is reconstituted and stored at 25° C. over a period of 24 hours, at most 0.4%, 0.3%, 0.2% or 0.1% of the voriconazole degrades.

A solid composition including voriconazole, HPCD, and an amino acid or disaccharide may be prepared by forming a liquid mixture that includes a solvent, voriconazole, HPCD, the amino acid or disaccharide, and optionally one or more other substances, and lyophilizing the liquid mixture. The lyophilizing may include freeze-drying the liquid mixture to provide a solid composition. The liquid mixture may include voriconazole, HPCD, and the amino acid or disaccharide in the amounts described above. The liquid mixture may further include a pH modifier and/or one or more other substances, as described above.

The liquid mixture may include from 5 to 50 mL solvent, from 50 to 500 mg voriconazole, from 1 to 4 g HPCD and from 250 mg to 2 g of the amino acid or disaccharide, and the liquid mixture is adjusted to a pH of from 5 to 7. The liquid mixture may include from 10 to 45 mL solvent, from 100 to 450 mg voriconazole, from 2 to 3.5 g HPCD and from 500 mg to 1.5 g of the amino acid or disaccharide, and the liquid mixture is adjusted to a pH of from 5.5 to 6.7. The liquid mixture may include from 15 to 25 mL solvent, from 150 to 250 mg voriconazole, from 2.5 to 3.0 g HPCD and from 900 mg to 1 g of the amino acid or disaccharide, and the liquid mixture is adjusted to a pH of from 6.0 to 6.7. In one example, the liquid mixture includes 20 mL water, 200 mg voriconazole, 2.7 g HPCD and 1 g arginine, and the liquid mixture is adjusted to a pH of 6.2 using HCl and NaOH.

The solvent, voriconazole, HPCD, the amino acid or disaccharide, and one or more other optional substances such as a pH modifier may be combined in any order when forming the liquid mixture. In one example, a liquid mixture may be formed by adding the voriconazole, the HPCD and the amino acid or disaccharide to a container including the solvent, and then adding the pH modifier to achieve the desired pH in the liquid mixture. In another example, a liquid mixture may be formed by combining the HPCD, the amino acid or disaccharide, and the solvent in a container, adding a pH modifier to achieve a first desired pH, adding the voriconazole to the container, and adding a pH modifier to achieve a final desired pH in the liquid mixture. In some embodiments, the liquid mixture comprises a pH modifier in an amount sufficient to provide a pH of from 5 to 7, from 5.5 to 6.7, or from 6.0 to 6.7 prior to lyophilization. In one example, the liquid mixture comprises a pH modifier in an amount sufficient to provide a pH of 6.2 prior to lyophilization.

The liquid mixture including the solvent, voriconazole, HPCD, the amino acid or disaccharide, and any other optional ingredients may be lyophilized to form a solid composition, such as by subjecting the liquid mixture to freeze-drying. Freeze-drying of the liquid mixture may include maintaining the liquid mixture in an inert atmosphere, such as nitrogen or argon. Preferably the liquid mixture is placed in glass vials prior to lyophilization, and the amount of the liquid mixture in each vial is based on the amount of voriconazole intended to be present in the final solid composition in the vial.

In a typical lyophilization process, the temperature of the liquid mixture is lowered to a temperature at or below the solidification point of the liquid mixture. If the liquid mixture forms a glass when cooled, the solidification point is the glass transition temperature. If the liquid mixture forms crystals when cooled, the solidification point is the eutectic point. The solidified mixture is then dried under vacuum. Typically, the drying process includes a primary drying step in which the temperature of the solidified mixture is raised gradually while most of the water is removed from the mixture by the vacuum, and a secondary drying step in which the temperature of the solidified mixture is raised further while residual moisture is removed from the mixture by the vacuum. The temperature is kept at or below the desired storage temperature for the final solid composition. Lyophilization typically is complete within 48 hours, but may require additional time. The solid composition resulting from the lyophilization typically is sealed for later use. Details regarding the lyophilization process may be found, for example, in Remington: The Science and Practice of Pharmacy, 20th Ed., ed. A. Gennaro, Lippincott Williams & Wilkins, 2000.

Lyophilizing the liquid mixture including the solvent, voriconazole, HPCD, the amino acid or disaccharide, and any other optional ingredients may include freezing the mixture at a temperature of about −45° C., and drying the liquid mixture at a temperature of from −35° C. to −15° C. and a pressure of from 50-200 millitorr (mTorr). The drying may be carried out at a temperature of from −32° C., −25° C. or −20° C. to −15° C., and at a pressure of 75 mTorr, 85 mTorr or 105 mTorr. The drying may be carried out for about 8 days or less, for about 12 days, or for about 21 days.

The lyophilized solid composition may be stored for later reconstitution and administration. Preferably the solid composition is stored at a temperature of from 10° C. to 40° C., from 15° C. to 35° C., from 20° C. to 30° C., or about 25° C. Preferably the solid composition is sealed in the glass vial to protect the composition from moisture in the surrounding environment.

A solid composition including voriconazole, HPCD, and an amino acid or disaccharide may be administered to a patient by combining the composition with an aqueous carrier liquid to form an aqueous mixture, and administering the aqueous mixture into the patient by, for example, injection. Preferably, the aqueous carrier liquid is a pharmaceutically acceptable carrier liquid. Non-limiting examples of pharmaceutically acceptable carrier liquids include water and saline, such as sodium chloride injection, phosphate buffered saline (PBS), Ringers solution or lactated Ringers injection. The aqueous carrier liquid also may include fixed oils, fatty esters or polyols, particularly if the aqueous mixture for injection is a suspension. The aqueous carrier liquid also may include one or more other substances such as buffers, stabilizers, solubilizers, preservatives and antioxidants. Preferably the solid composition dissolves in the aqueous carrier liquid to form a solution.

Presently preferred aqueous carrier liquids include sodium chloride injection, such as solutions containing 0.9%, 0.45% or 0.225% sodium chloride. Presently preferred aqueous carrier liquids include sterile water for injection. Presently preferred aqueous carrier liquids include bacteriostatic water for injection, which may include, for example, either 0.9% benzyl alcohol or a combination of methylparaben and propylparaben. Presently preferred aqueous carrier liquids include lactated Ringers injection.

The amount of aqueous carrier liquid may be sufficient to provide an initial aqueous mixture containing voriconazole at a concentration of 10 mg/mL. At this concentration, it is convenient to provide a 200 mg dose of voriconazole to a patient, such as by dispensing 20 mL of the mixture into another aqueous liquid to form a final mixture for administration. While an initial aqueous mixture containing voriconazole at a concentration of 10 mg/mL may be injected into a patient, the presently recommended procedure includes combining the initial mixture with another aqueous liquid to form a final aqueous mixture having a voriconazole concentration of 5 mg/mL, which is then administered to a patient.

The amount of aqueous carrier liquid may be sufficient to provide a final aqueous mixture containing voriconazole at a concentration of at most 5 mg/mL. For example, 20 mL of an initial aqueous mixture containing 10 mg/mL voriconazole may be combined with 20 mL of an aqueous carrier liquid to provide a final aqueous mixture containing about 5 mg/mL voriconazole. Presently preferred concentrations of voriconazole in a final aqueous mixture for administration to a patient are from 4 to 6 mg/mL, including 4.5 to 5.5 mg/mL and 4.9 to 5.1 mg/mL.

An aqueous mixture formed from the solid composition may be administered to provide an initial dose to a patient of 6 mg voriconazole per kilogram of patient body weight (6 mg/kg). This initial dose may be administered every 12 hours for the first day of treatment. An aqueous mixture formed from the solid composition may be administered to provide a maintenance dose of voriconazole to a patient of 3-4 mg/kg, which may be administered to the patient twice a day. Doses outside of these ranges also may be administered. Typically, an initial dose of voriconazole includes 6 mg/kg, and subsequent maintenance doses include 3-4 mg/kg. Higher maintenance doses than 3-4 mg/kg may be advisable under certain conditions, such as an insufficient response by the fungal infection. Maintenance doses of voriconazole below 3-4 mg/kg may be advisable under certain conditions, such as for pediatric patients or patients having moderate hepatic impairment.

A composition of the invention can be administered as a monotherapy, or a composition of the invention can be a component of a combination therapy comprising the administration of voriconazole and one or more additional drugs. If a component of a combination therapy, a composition of the invention can be administered prior to, substantially concurrent with, or after the administration of the one or more additional drugs.

The following examples are intended to illustrate the invention in a non-limiting manner.

### Example 1

This example demonstrates the effect of HPCD or SBECD on the stability of voriconazole in a lyophilized composition.

Lyophilized compositions were formed by dissolving the excipient (SBECD or HPCD) in water and adjusting the pH of the resulting excipient solution. Voriconazole (200 mg) was added to the excipient solution, and the pH of the resulting lyophilization solution was adjusted as necessary. The lyophilization solutions were then filtered and lyophilized to form solid compositions. The lyophilization procedure was adjusted as need for each type of composition; however, the general procedure included reducing the temperature of the lyophilization solutions to −45° C., performing a primary drying at a temperature of from −35 to −15° C. and under a vacuum of from 50-200 milliTorr (mTorr), and performing a secondary drying at a temperature of 40° C. To ensure the lowest initial impurities possible, the solutions for lyophilization were prepared at low temperature, using a nitrogen blanket and purging.

Each composition was sealed in a 5 mL vial with a 13 mm rubber stopper (STELMI 6720GC; American Stelmi Corporation, Princeton, N.J.) and stored at 25° C., 40° C. or 55° C. (all temperatures ±2° C.). After storage for 4 or 8 weeks at 25° C., for 4, 8 or 12 weeks at 40° C., or for 2 or 4 weeks at 55° C., a portion of the vials were reconstituted with 19 mL of water for injection. The reconstituted liquids were analyzed by HPLC to determine the concentrations of voriconazole and of any impurities. For comparison, a portion of each composition was reconstituted at the beginning of the storage time (time=0 weeks) with 19 mL of water for injection, and analyzed by HPLC for voriconazole and impurity concentrations.

Table 1 lists the results of stability analyses of voriconazole alone, or of lyophilized compositions containing voriconazole in combination with either SBECD or HPCD as solubilizers.

TABLE 1
Stability of voriconazole with and without a cyclodextrin.

| | Amount | | | | |
|---|---|---|---|---|---|
| | (g) per | | | | |
| | 200 mg | pH at | Storage | | Total |
| | vori- | lyoph- | temp | Time | impurities |
| Excipient | conazole | ilization | (° C.) | (weeks) | (%) |
| — | — | — | — | 0 | 0.18 |
| | | | 40 | 4 | 0.70 |
| | | | | 8 | 0.34 |
| | | | | 12 | 0.43 |
| | | | 55 | 2 | 0.58 |
| SBECD* | 3.2 | 6.12 | — | 0 | 0.06 |
| | | | 25 | 4 | 0.07 |
| | | | | 8 | 0.08 |
| | | | 40 | 4 | 0.24 |
| | | | | 8 | 0.40 |
| | | | | 12 | 0.47 |
| | | | 55 | 2 | 0.51 |
| | | | | 4 | 0.81 |
| HPCD** | 2.7 | 5.18 | — | 0 | 0.07 |
| | | | 25 | 4 | 0.12 |
| | | | | 8 | 0.18 |
| | | | 40 | 4 | 0.73 |
| | | | | 8 | 1.48 |
| | | | | 12 | 1.94 |
| | | | 55 | 2 | 1.86 |
| | | | | 4 | 3.26 |

*Sulfobutyl ether β-cyclodextrin sodium (Captisol)
**Hydroxypropyl β-cyclodextrin (Kleptose)

The presence of a cyclodextrin solubilizer affected the stability of the voriconazole in the resulting lyophilized compositions. The lyophilized solids containing SBECD solubilizer had lower levels of impurities, relative to voriconazole alone, over time for each temperature studied. In contrast, the lyophilized solids containing HPCD solubilizer had higher levels of impurities, relative to voriconazole alone, over time for each temperature studied. Accordingly, replacing SBECD with HPCD did not improve the stability of voriconazole in a solid composition.

### Example 2

This example demonstrates the effect of solution pH on the stability of a lyophilized composition comprising voriconazole and HPCD.

Solutions comprising HPCD and voriconazole were prepared as described in Example 1, except that the pH of the solution prior to lyophilization was adjusted to 5.18, 5.5, 6.0, 6.5, or 7.0. The lyophilized compositions were formed and then analyzed for the stability of voriconazole over time at 40° C. or 55° C., as described above with regard to Table 1. Table 2 lists the results of stability analyses of lyophilized compositions containing voriconazole in combination with HPCD, where the compositions had different pH values prior to lyophilization. The entries for compositions having a pH of 5.18 prior to lyophilization are the same as those listed in Table 1.

TABLE 2
Stability of voriconazole in lyophilized
formulations with HPCD (2.7 g
HPCD/200 mg voriconazole).

| Storage | | pH at | Total |
|---|---|---|---|
| temp | Time | lyoph- | impurities |
| (° C.) | (weeks) | ilization | (%) |
| — | 0 | — | 0.07 |
| 40 | 4 | 5.18 | 0.73 |
| | | 5.5 | 0.67 |
| | | 6.0 | 0.73 |
| | | 6.5 | 0.92 |
| | | 7.0 | 1.50 |
| 55 | 2 | 5.18 | 1.86 |
| | | 5.5 | 3.69 |
| | | 6.0 | 3.96 |
| | | 6.5 | 4.33 |
| | | 7.0 | 6.25 |

The pH of the solutions prior to lyophilization affected the stability of the voriconazole in the resulting solid compositions. For the temperatures studied, the concentration of impurities was lower in compositions formed from solutions having acidic pH levels from 5.18 to 6.0, and was higher in compositions formed from solutions having a neutral pH.

### Example 3

This example demonstrates the effect of an amino acid on the stability of a lyophilized composition comprising voriconazole and HPCD.

Solutions comprising HPCD and voriconazole were prepared as described in Example 1, except that the solutions additionally contained arginine, aspartic acid or glycine. In forming the compositions, the amino acid was present in the excipient solution, to which the voriconazole was added. Each amino acid was present at a level of 500 mg per 200 mg voriconazole.

For arginine, 500 mg arginine per 200 mg voriconazole corresponds to a molar ratio of amino acid to voriconazole of about 5:1 (0.0029 moles:0.00057 moles=[0.5 g arginine÷(174.20 g arginine/mol)]:[0.2 g voriconazole÷(349.31 g voriconazole/mol)]). For aspartic acid, 500 mg aspartic acid per 200 mg voriconazole corresponds to a molar ratio of amino acid to voriconazole of about 6.6:1 (0.0038 moles:0.00057 moles=[0.5 g aspartic acid÷(133.10 g aspartic acid/mol)]: [0.2 g voriconazole÷(349.31 g voriconazole/mol)]). For glycine, 500 mg glycine per 200 mg voriconazole corresponds to a molar ratio of amino acid to voriconazole of about 11.6:1 (0.0067 moles:0.00057 moles=[0.5 g glycine÷(75.07 g glycine/mol)]: [0.2 g voriconazole÷(349.31 g voriconazole/mol)]).

The pH prior to lyophilization was adjusted, using one of a variety of pH modifiers, as listed in Table 3. The lyophilized compositions were formed and then analyzed for voriconazole stability over time at 40° C. and 55° C., as described above with regard to Table 1. Table 3 lists the results of stability analyses of lyophilized compositions containing voriconazole in combination with HPCD, where the compositions further included one of the amino acids arginine, aspartic acid or glycine.

TABLE 3
Stability of voriconazole in lyophilized formulations
with HPCD (2.7 g/200 mg voriconazole)
and arginine, aspartic acid or glycine.

| | pH at | | Storage | | Total |
|---|---|---|---|---|---|
| Amino | lyoph- | pH | temp | Time | impurities |
| acid* | ilization | modifier | (° C.) | (weeks) | (%) |
| Arginine | 6.4 | HCl/NaOH | — | 0 | 0.02 |
| | | | 40 | 4 | 0.30 |
| | | | | 6 | 0.39 |
| | | | | 8 | 0.48 |
| | | | 55 | 2 | 0.82 |
| | | | | 4 | 1.56 |
| | | NH₄OH/ | — | 0 | 0.11 |
| | | Acetic acid | 55 | 2 | 2.13 |
| | 10 | HCl/NaOH | — | 0 | 3.60 |
| | | | 55 | 2 | 43.11 |
| Aspartic acid | 6.4 | HCl/NaOH | — | 0 | 0.13 |
| | | | 40 | 4 | 1.06 |
| | | | | 8 | 1.78 |
| | | | 55 | 2 | 3.12 |
| | | | | 4 | 6.13 |
| | | NH₄OH/ | — | 0 | 0.06 |
| | | Acetic acid | 55 | 2 | 3.50 |
| | 3 | HCl/NaOH | — | 0 | — |
| | | | 55 | 2 | 3.32 |
| Glycine | 6.4 | HCl/NaOH | — | 0 | 0.11 |
| | | | 40 | 4 | 0.85 |
| | | | | 8 | 1.54 |
| | | | 55 | 2 | 2.89 |
| | | | | 4 | 5.96 |
| | | NH₄OH/ | — | 0 | 0.05 |
| | | Acetic acid | 55 | 2 | 3.80 |
| | 6 | HCl/NaOH | — | 0 | 0.05 |
| | | | 55 | 2 | 3.77 |

*500 mg amino acid/200 mg voriconazole

The solid voriconazole compositions containing HPCD and arginine provided better stability than the voriconazole compositions containing HPCD and glycine. The total impurities measured for the arginine compositions were lower than those measured for the glycine compositions by a factor of about 3 or 4. For example, the composition containing HPCD and arginine and having a pH prior to lyophilization of 6.4 as modified by HCl/NaOH had a total impurity level of 0.30% after 4 weeks at 40° C. The comparable composition containing HPCD and glycine had a total impurity level after 4 weeks at 40° C. of 0.85%, which was nearly 3 times that of the arginine composition (2.83=0.85%/0.30%). Similarly, the composition containing HPCD and arginine and having a pH prior to lyophilization of 6.4 as modified by HCl/NaOH had a total impurity level of 0.82% after 2 weeks at 55° C. The comparable composition containing HPCD and glycine had a total impurity level after 2 weeks at 55° C. of 2.89%, which was about 3.5 times that of the arginine composition (3.52=2.89%/0.82%).

In the solid voriconazole compositions containing HPCD and arginine, the pH modifier of HCl with NaOH provided better stability of voriconazole than the pH modifier of NH₄OH with acetic acid. In addition, arginine compositions lyophilized from a liquid having an acidic pH of 6.4 provided better stability of voriconazole than the compositions lyophilized from a liquid having a basic pH of 10.

Solutions comprising HPCD and voriconazole also were prepared containing lysine, histidine, asparagine, glutamine or threonine. Two types of lysine-containing compositions were formed, where one type of composition included 1 g lysine per 200 mg voriconazole, and the other type included 500 mg lysine per 200 mg voriconazole. Each of the amino acids histidine, asparagine, glutamine and threonine was present at a level of 500 mg per 200 mg voriconazole.

For lysine, 500 mg lysine per 200 mg voriconazole corresponds to a molar ratio of amino acid to voriconazole of about 6:1 (0.0034 moles:0.00057 moles=[0.5 g lysine÷(146.19 g lysine/mol)]: [0.2 g voriconazole÷(349.31 g voriconazole/mol)]). In addition, 1 g lysine per 200 mg voriconazole corresponds to a molar ratio of amino acid to voriconazole of about 12:1 (0.0068 moles:0.00057 moles=[1 g lysine÷(146.19 g lysine/mol)]:[0.2 g voriconazole÷(349.31 g voriconazole/mol)]).

For histidine, 500 mg histidine per 200 mg voriconazole corresponds to a molar ratio of amino acid to voriconazole of about 5.6:1 (0.0032 moles:0.00057 moles=[0.5 g histidine÷(155.15 g histidine/mol)]:[0.2 g voriconazole÷(349.31 g voriconazole/mol)]). For asparagine, 500 mg asparagine per 200 mg voriconazole corresponds to a molar ratio of amino acid to voriconazole of about 6.6:1 (0.0038 moles:0.00057 moles=[0.5 g asparagine÷(132.12 g asparagine/mol)]:[0.2 g voriconazole÷(349.31 g voriconazole/mol)]). For glutamine, 500 mg glutamine per 200 mg voriconazole corresponds to a molar ratio of amino acid to voriconazole of about 6:1 (0.0034 moles:0.00057 moles=[0.5 g glutamine÷(146.14 g glutamine/mol)]: [0.2 g voriconazole÷(349.31 g voriconazole/mol)]). For threonine, 500 mg threonine per 200 mg voriconazole corresponds to a molar ratio of amino acid to voriconazole of about 7.3:1 (0.0042 moles:0.00057 moles=[0.5 g threonine÷(119.12 g threonine/mol)]:[0.2 g voriconazole÷(349.31 g voriconazole/mol)]).

The pH prior to lyophilization was adjusted, using one of a variety of pH modifiers, as listed in Table 4. The lyophilized compositions were formed and then analyzed for voriconazole stability over time at 40° C. and 55° C., as described above with regard to Table 3. Table 4 lists the results of stability analyses of lyophilized compositions containing voriconazole in combination with HPCD, where the compositions further included one of the amino acids lysine, histidine, asparagine, glutamine or threonine.

TABLE 4
Stability of lyophilized voriconazole with
HPCD (2.7 g/200 mg voriconazole) and lysine,
histidine, asparagine, glutamine or threonine.

| | pH at | | Storage | | Total |
|---|---|---|---|---|---|
| Amino | lyoph- | pH | temp | Time | impurities |
| acid* | ilization | modifier | (° C.) | (weeks) | (%) |
| Lysine | 5.5 | HCl/NaOH | — | 0 | 0.07 |
| | | | 55 | 2 | 1.60 |
| | 6.4 | HCl/NaOH | — | 0 | 0.20 |
| | | | 55 | 2 | 1.60 |
| | | NH₄OH/ | — | 0 | 0.02 |
| | | Acetic acid | 55 | 2 | 1.69 |
| | | Citric acid | — | 0 | 0.66 |
| | | | 55 | 2 | 4.90 |
| Lysine | 6.4 | HCl | — | 0 | 0.40 |
| (1 g/200 mg | | | 40 | 4 | 0.64 |
| voriconazole) | | | 55 | 2 | 1.00 |
| | | HCl/NaOH | — | 0 | 0.40 |
| | | | 40 | 4 | 0.60 |
| | | | 55 | 2 | 1.00 |
| | | Citrate | — | 0 | 1.10 |
| | | | 40 | 4 | 2.70 |
| | | | 55 | 2 | 4.80 |
| Histidine | 7.5 | HCl/NaOH | — | 0 | 0.29 |
| | | | 55 | 2 | 2.82 |
| | 6.4 | NH₄OH/ | — | 0 | 0.26 |
| | | Acetic acid | 55 | 2 | 2.41 |
| Asparagine | 6.4 | Citrate | — | 0 | 0.30 |
| | | | 55 | 2 | 2.20 |
| | | HCl | — | 0 | 0.08 |
| | | | 55 | 2 | 2.20 |
| Glutamine | 6.4 | HCl/NaOH | — | 0 | 0.10 |
| | | | 40 | 4 | 1.90 |
| Threonine | 6.4 | HCl/NaOH | — | 0 | 0.14 |
| | | | 40 | 4 | 0.16 |
| | | | 55 | 2 | 0.70 |

*500 mg amino acid/200 mg voriconazole, unless otherwise noted

The solid voriconazole compositions containing HPCD and either lysine or threonine provided better stability than the voriconazole compositions containing HPCD and glycine (Table 3). The total impurities measured for the arginine compositions were lower than those measured for the glycine compositions by a factor of about 2 to 4. For example, the composition containing HPCD and lysine and having a pH prior to lyophilization of 6.4 as modified by HCl/NaOH had total impurity levels of 1.60% or 1.00% after 2 weeks at 55° C. The comparable composition containing HPCD and glycine had a total impurity level after 2 weeks at 55° C. of 2.89%, which was about 2 to 3 times that of the lysine composition (1.81=2.89%/1.60%; 2.89=2.89%/1.00%). Similarly, the composition containing HPCD and threonine and having a pH prior to lyophilization of 6.4 as modified by HCl/NaOH had a total impurity level of 0.70% after 2 weeks at 55° C. The comparable composition containing HPCD and glycine had a total impurity level after 2 weeks at 55° C. of 2.89%, which was about 4 times that of the threonine composition (4.12=2.89%/0.70%).

The results of this example demonstrate that arginine, lysine, or threonine stabilize voriconazole in compositions comprising HPCD as a solubilizer.

### Example 4

This example demonstrates the effect of a non-amino acid excipient on the stability of a lyophilized composition comprising voriconazole and HPCD.

Solutions comprising HPCD and voriconazole were prepared as described in Example 1, except that the solutions additionally contained PEG 1000, dextran, lactose, tris maleate or trehalose. The lyophilized compositions were formed as described above with regard to Table 3, with the excipient present in the excipient solution, to which the voriconazole was added. Each excipient was present at a level of 500 mg per 200 mg voriconazole.

For lactose, 500 mg lactose per 200 mg voriconazole corresponds to a molar ratio of excipient to voriconazole of about 2.6:1 (0.0015 moles:0.00057 moles=[0.5 g lactose÷(342.30 g lactose/mol)]:[0.2 g voriconazole÷(349.31 g voriconazole/mol)]). For tris maleate, 500 mg tris maleate per 200 mg voriconazole corresponds to a molar ratio of excipient to voriconazole of about 3.7:1 (0.0021 moles: 0.00057 moles=[0.5 g tris maleate÷(237.21 g tris maleate/mol)]:[0.2 g voriconazole÷(349.31 g voriconazole/mol)]). For trehalose, 500 mg trehalose per 200 mg voriconazole corresponds to a molar ratio of excipient to voriconazole of about 2.6:1 (0.0015 moles:0.00057 moles=[0.5 g trehalose÷(342.30 g trehalose/mol)]: [0.2 g voriconazole÷(349.31 g voriconazole/mol)]).

The effects of pH prior to lyophilization were analyzed for the formulations that included lactose. The pH prior to lyophilization was adjusted, as listed in Table 5. The lyophilized compositions were formed and then analyzed for voriconazole stability over time at 40° C. and 55° C., as described above with regard to Table 3. Table 5 lists the results of stability analyses of lyophilized compositions containing voriconazole in combination with HPCD solubilizer, where the compositions further included one of the non-amino acid excipients PEG 1000, dextran, lactose, tris maleate or trehalose.

TABLE 5
Stability of lyophilized voriconazole with
HPCD (2.7 g/200 mg voriconazole)
and non-amino acid excipients.

| | pH at | Storage | | Total |
|---|---|---|---|---|
| | lyoph- | temp | Time | impurities |
| Excipient | ilization | (° C.) | (weeks) | (%) |
| PEG 1000 | 6.4 | — | 0 | 0.04 |
| | | 40 | 4 | 1.42 |
| | | 55 | 2 | 4.1 |
| Dextran | 6.4 | — | 0 | 0 |
| | | 40 | 4 | 0.77 |
| | | 55 | 2 | 2.94 |
| Lactose | 4.6 | — | 0 | 0 |
| | | 40 | 4 | 0.26 |
| | | 55 | 2 | 0.9 |
| | 6.4 | — | 0 | 0 |
| | | 40 | 4 | 0.44 |
| | | 55 | 2 | 1.2 |
| Tris maleate | 6.4 | — | 0 | 17.3 |
| | | 55 | 2 | 21.2 |
| Trehalose | 6.2 | — | 0 | 0.02 |
| | | 40 | 4 | 0.29 |
| | | 55 | 2 | 0.70 |

The solid voriconazole compositions containing HPCD and either lactose or trehalose as a disaccharide provided better stability than the voriconazole compositions containing HPCD and glycine (Table 3). The total impurities measured for the arginine compositions were lower than those measured for the glycine compositions by a factor of about 2 to 4. For example, the composition containing HPCD and lactose and having a pH prior to lyophilization of 6.4 as modified by HCl/NaOH had a total impurity level of 1.2% after 2 weeks at 55° C. The comparable composition containing HPCD and glycine had a total impurity level after 2 weeks at 55° C. of 2.89%, which was about 2.4 times that of the lactose composition (2.41=2.89%/1.2%). Similarly, the composition containing HPCD and trehalose and having a pH prior to lyophilization of 6.2 as modified by HCl/NaOH had a total impurity level of 0.70% after 2 weeks at 55° C. The comparable composition containing HPCD and glycine had a total impurity level after 2 weeks at 55° C. of 2.89%, which was about 4 times that of the trehalose composition (4.12=2.89%/0.70%).

The results of this example demonstrate that voriconazole in solid compositions that include HPCD and an excipient selected from lysine, threonine, lactose and trehalose may provide stability to voriconazole that is better than the stability provided by solid compositions that include HPCD and glycine. Solid compositions having a molar ratio of voriconazole to HPCD of 1:3, having a molar ratio of voriconazole to glycine of 1:11.6, and having a pH of 6.4 prior to lyophilization had impurity levels of 0.85% after 4 weeks of storage at 40° C., and had impurity levels of 2.89% after 2 weeks of storage at 55° C. See Table 3, above. In contrast, solid compositions including lysine, threonine, lactose or trehalose, and having a molar ratio of voriconazole to HPCD of 1:3, had lower impurity levels of at most 0.64% after 4 weeks of storage at 40° C., and had lower impurity levels of at most 1.69% after 2 weeks of storage at 55° C. See Tables 4 and 5, above. Thus, these solid voriconazole compositions that included HPCD and lysine, threonine, lactose or trehalose had voriconazole stability levels that were higher than those provided by voriconazole compositions that included HPCD and glycine.

### Example 5

This example demonstrates the effect of varying amounts of arginine and HPCD on the stability of a lyophilized voriconazole composition.

Solutions comprising HPCD, arginine, and voriconazole were prepared as described in Example 3, except that the molar ratio of voriconazole to HPCD was varied from 1:3 to 1:5.5, and the molar ratio of voriconazole to arginine was varied from 1:2.5 to 1:20.

For the compositions containing 250 mg arginine per 200 mg voriconazole, the molar ratio of voriconazole to amino acid was about 1:2.5 (0.00057 moles:0.0014 moles=[0.2 g voriconazole÷(349.31 g voriconazole/mol)]:[0.25 g arginine÷(174.20 g arginine/mol)]). For the compositions containing 500 mg arginine per 200 mg voriconazole, the molar ratio of voriconazole to amino acid was about 1:5 (0.00057 moles:0.0029 moles=[0.2 g voriconazole÷(349.31 g voriconazole/mol)]:[0.5 g arginine÷(174.20 g arginine/mol)]). For the compositions containing 1 g arginine per 200 mg voriconazole, the molar ratio of voriconazole to amino acid was about 1:10 (0.00057 moles:0.0057 moles=[0.2 g voriconazole÷(349.31 g voriconazole/mol)]:[1 g arginine÷(174.20 g arginine/mol)]). For the compositions containing 1.5 g arginine per 200 mg voriconazole, the molar ratio of voriconazole to amino acid was about 1:15 (0.00057 moles:0.0086 moles=[0.2 g voriconazole÷(349.31 g voriconazole/mol)]:[1.5 g arginine÷(174.20 g arginine/mol)]). For the compositions containing 2 g arginine per 200 mg voriconazole, the molar ratio of voriconazole to amino acid was about 1:20 (0.00057 moles:0.0115 moles=[0.2 g voriconazole÷(349.31 g voriconazole/mol)]:[2 g arginine÷(174.20 g arginine/mol)]).

The pH prior to lyophilization was adjusted to 6.2 with HCl/NaOH. The lyophilized compositions were formed and then analyzed for voriconazole stability over time at 40° C. and 55° C., as described above with regard to Table 3. Table 6 lists the results of stability analyses of lyophilized compositions containing voriconazole in combination with HPCD and arginine, where the molar ratio of voriconazole to HPCD was varied from 1:3 to 1:5.5, and the molar ratio of voriconazole to arginine was varied from 1:2.5 to 1:20.

TABLE 6
Stability of lyophilized voriconazole with
HPCD, arginine and HCl/NaOH.

| Molar ratio of | | Storage | | Total |
|---|---|---|---|---|
| voriconazole to . . . | | temp | Time | impurities |
| HPCD | Arginine | (° C.) | (weeks) | (%) |
| 1:3 | 2.5 | — | 0 | 0.10 |
| | | 40 | 4 | 0.40 |
| | | 55 | 2 | 1.20 |
| | 5 | — | 0 | 0.10 |
| | | 40 | 4 | 0.30 |
| | | 55 | 2 | 0.70 |
| | 10 | — | 0 | 0.10 |
| | | 40 | 4 | 0.20 |
| | | 55 | 2 | 0.40 |
| | 15 | — | 0 | 0.20 |
| | | 40 | 4 | 0.30 |
| | | 55 | 2 | 0.32 |
| | 20 | — | 0 | 0.20 |
| | | 40 | 4 | 0.30 |
| | | 55 | 2 | 0.30 |
| 1:4 | 5 | — | 0 | 0.11 |
| | | 55 | 2 | 1.00 |
| | 10 | — | 0 | 0.15 |
| | | 55 | 2 | 0.50 |
| 1:5.5 | 2.5 | — | 0 | 0.1 |
| | | 40 | 4 | 0.69 |
| | | 55 | 2 | 1.9 |
| | 5 | — | 0 | 0.1 |
| | | 40 | 4 | 0.4 |
| | | 55 | 2 | 1.3 |
| | 10 | — | 0 | 0.2 |
| | | 40 | 4 | 0.3 |
| | | 55 | 2 | 0.7 |

Thus, solid compositions having a molar ratio of voriconazole to HPCD of from 1:3 to 1:5.5, having a molar ratio of voriconazole to arginine of 1:2.5 to 1:20, and having a pH of 6.2 prior to lyophilization had lower impurity levels of at most 0.69% after 4 weeks of storage at 40° C., and had lower impurity levels of at most 1.9% after 2 weeks of storage at 55° C. See Table 6, above.

These results demonstrate that voriconazole in solid compositions that include HPCD and arginine may provide stability to voriconazole that is similar to or better than the stability provided by solid compositions that include SBECD. Solid compositions having a molar ratio of voriconazole to HPCD of 1:3 or 1:4, and having a molar ratio of voriconazole to arginine of 1:10 to 1:20, had impurity levels of from 0.30 to 0.50% after 2 weeks of storage at 55° C. See Table 6, above. In comparison, solid compositions of voriconazole with SBECD had higher impurity levels of 0.51% after 2 weeks of storage at 55° C. See Table 1, above. Thus, these solid voriconazole compositions that included HPCD and the amino acid arginine had voriconazole stability levels that were higher than those provided by conventional voriconazole compositions that included SBECD.

Similar comparative results were observed at 40° C. Solid compositions having a molar ratio of voriconazole to HPCD of 1:3, and having a molar ratio of voriconazole to arginine of from 1:5 to 1:20, had an impurity level of from 0.20 to 0.30% after 4 weeks of storage at 40° C. Solid compositions having a molar ratio of voriconazole to HPCD of 1:5.5, and having a molar ratio of voriconazole to arginine of 1:10, had an impurity level of 0.30% after 4 weeks of storage at 40° C. See Table 6, above. In comparison, solid compositions of voriconazole with SBECD had impurity levels of 0.24% after 4 weeks of storage at 40° C. See Table 1, above. Thus, conventional voriconazole compositions that included SBECD had voriconazole stability levels within the range of stability provided by these solid voriconazole compositions that included HPCD and the amino acid arginine.

### Example 6

This example demonstrates the effect of varying amounts of arginine and HPCD, type of pH modifier, and/or presence of buffer on the stability of a lyophilized voriconazole composition.

Solutions comprising HPCD, arginine, and voriconazole were prepared as described in Example 5, except that a pH modifier other than HCl/NaOH was used. The molar ratio of voriconazole to HPCD was varied from 1:2.5 to 1:5.5, and the molar ratio of voriconazole to arginine was varied from 1:5 to 1:10. The lyophilized compositions were formed and then analyzed for voriconazole stability over time at 40° C. and 55° C., as described above with regard to Table 3. The pH prior to lyophilization was adjusted to 6.2 with NH₄OH/acetic acid. Table 7 lists the results of stability analyses of lyophilized compositions containing voriconazole in combination with HPCD and arginine using NH₄OH/acetic acid as the pH modifier.

TABLE 7
Stability of voriconazole with HPCD,
arginine and NH₄OH/acetic acid.

| Molar ratio of | | Storage | | Total |
|---|---|---|---|---|
| voriconazole to . . . | | temp | Time | impurities |
| HPCD | Arginine | (° C.) | (weeks) | (%) |
| 1:2.5 | 5 | — | 0 | 0.12 |
| | | 55 | 2 | 1.62 |
| | 10 | — | 0 | 0.20 |
| | | 55 | 2 | 0.93 |
| 1:3 | 5 | — | 0 | 0.13 |
| | | 40 | 4 | 0.62 |
| | | 55 | 2 | 1.94 |
| | 10 | — | 0 | 0.21 |
| | | 40 | 4 | 0.47 |
| | | 55 | 2 | 1.08 |
| 1:3.5 | 5 | — | 0 | 0.15 |
| | | 40 | 4 | 0.78 |
| | | 55 | 2 | 2.43 |
| | 10 | — | 0 | 0.22 |
| | | 40 | 4 | 0.51 |
| | | 55 | 2 | 1.19 |
| 1:3.9 | 5 | — | 0 | 0.14 |
| | | 55 | 2 | 2.46 |
| | 10 | — | 0 | 0.20 |
| | | 55 | 2 | 1.39 |
| 1:5.5 | 5 | — | 0 | 0.17 |
| | | 55 | 2 | 3.00 |
| | 10 | — | 0 | 0.23 |
| | | 55 | 2 | 1.80 |

Table 8 lists the results of stability analyses of lyophilized compositions containing voriconazole in combination with HPCD and arginine, but using different pH modifiers than the HCl/NaOH pH modifier listed in Table 6 or the NH₄OH/acetic acid pH modifier listed in Table 7. The molar ratio of voriconazole to HPCD was varied from 1:3 to 1:5, and the molar ratio of voriconazole to arginine was varied from 1:5 to 1:10. The lyophilized compositions were formed and then analyzed for voriconazole stability over time at 40° C. and 55° C., as described above with regard to Table 3. The pH prior to lyophilization was adjusted to 6.2 with citric acid, tris maleate, citrate buffer or phosphate buffer.

TABLE 8
Stability of voriconazole with HPCD,
arginine and various buffers.

| Molar ratio of | | | Storage | | Total |
|---|---|---|---|---|---|
| voriconazole to ... | | | temp | Time | impurities |
| HPCD | Arginine | Buffer | (° C.) | (weeks) | (%) |
| 1:3 | 5 | Citric acid | — | 0 | 0.06 |
| | | | 40 | 4 | 0.64 |
| | | | 55 | 2 | 1.13 |
| | | Tris maleate | — | 0 | 15.30 |
| | | | 55 | 2 | 14.8 |
| 1:4 | 5 | Citric acid | — | 0 | 0.53 |
| | | | 55 | 2 | 3.8 |
| | 10 | Citrate | — | 0 | 0.8 |
| | | | 40 | 4 | 2.21 |
| | | | 55 | 2 | 2.7 |
| | | Phosphate | — | 0 | 0.1 |
| | | | 40 | 4 | 0.31 |
| | | | 55 | 2 | 0.5 |
| 1:5 | 5 | Citric acid | — | 0 | 0.6 |
| | | | 40 | 4 | 1.7 |
| | | | 55 | 2 | 3.6 |

The results of this example demonstrate that HCl, NaOH, NH₄OH, acetic acid, and phosphate are suitable pH adjusting agents/buffers for compositions comprising arginine, HPCD, and voriconazole.

### Example 7

This example compares the stability of reconstituted compositions comprising voriconazole, HPCD, and arginine to the stability of reconstituted compositions comprising voriconazole and SBECD.

Solutions containing voriconazole and SBECD or voriconazole, HPCD, and arginine were prepared as described in Examples 1 and 3. For the composition containing SBECD, the amount of cyclodextrin was 3.2 g per 200 mg voriconazole. For the compositions containing HPCD, the amount of cyclodextrin was 2.7 g per 200 mg voriconazole. For the compositions containing arginine, the arginine was present in the excipient solution, to which the voriconazole was added, and the pH prior to lyophilization was adjusted to 6.4 with HCl/NaOH. The lyophilized compositions were formed as described above with regard to Table 1.

Each lyophilized composition was reconstituted in 19 mL water for injection, sealed in a vial with a rubber stopper, and stored for 1 week at 0° C., 5° C., 25° C., 40° C. or 55° C. (all temperatures ±2° C.). After storage, each sample was analyzed by HPLC to determine the concentrations of voriconazole and of any impurities. Table 9 lists the results of stability analyses of reconstituted solutions of lyophilized compositions containing voriconazole.

TABLE 9
Reconstituted solution stability, for 1 week, of voriconazole
with a cyclodextrin, with or without arginine.

| | Molar ratio of | pH at | Storage | Total |
|---|---|---|---|---|
| Cyclo- | voriconazole | lyoph- | temp | impurities |
| dextrin | to arginine | ilization | (° C.) | (%) |
| SBECD | — | — | 0 | 0.09 |
| | | | 5 | 0.63 |
| | | | 25 | 0.18 |
| | | | 40 | 1.11 |
| | | | 55 | 9.15 |
| HPCD | — | — | 0 | 0 |
| | | | 5 | 0.01 |
| | | | 25 | 0.12 |
| | | | 40 | 1.14 |
| | 5 | 6.4 | 0 | 0.1 |
| | | | 5 | 0.13 |
| | | | 25 | 0.35 |
| | | | 40 | 1.59 |
| | | | 55 | 12.69 |
| | 10 | 6.4 | 0 | 0.17 |
| | | | 5 | 0.22 |
| | | | 25 | 0.24 |
| | | | 40 | 1.71 |

The results of this example demonstrate that reconstituted compositions comprising voriconazole, HPCD, and arginine can exhibit greater stability than reconstituted compositions comprising voriconazole and SBECD.

### Example 8

This example demonstrates the long-term storage stability of a composition of the invention.

A solution was prepared by dissolving HPCD (133 mg/mL) and arginine (50 mg/mL) in water. Voriconazole (10 mg/mL) was added, and the pH of the solution was adjusted to 6.2 with HCl and/or NaOH. The solution was filtered and filled into vials in an amount of 20 mL per vial. The filled vials were sealed with a rubber stopper, and lyophilized. Each vial contained approximately 200 mg voriconazole, 2660 mg HPCD, and 1000 mg arginine, providing a molar ratio of voriconazole to HPCD of approximately 1:3.25 and a molar ratio of voriconazole to arginine of approximately 1:10.

The vials containing the lyophilized composition were stored at 25±2° C. and 60±5% relative humidity (RH) or at 40±2° C. and 75±5% RH in an upright (↑) or inverted (↓) orientation for 1 month, 2 months, or 3 months. After storage, a portion of the vials were reconstituted with water, and the reconstituted liquids were analyzed by HPLC to determine the concentrations of voriconazole and of total impurities. For comparison, a portion of the lyophilized composition was reconstituted at the beginning of the storage time (time=0) and analyzed similarly. Table 10 lists the results of the stability analyses.

TABLE 10
Stability of composition comprising voriconazole
(200 mg), HPCD (2660 mg), and arginine (1000 mg)

| Storage duration | Total Impurities | |
|---|---|---|
| (vial orientation) | 25 ° C. | 40 ° C. |
| t = 0 | <0.025% | <0.025% |
| 1-month (↑) | 0.03% | 0.17% |
| 1-month (↓) | 0.03% | 0.08% |
| 2-months (↑) | <0.025% | 0.06% |
| 2-months (↓) | <0.025% | 0.06% |
| 3-months (↑) | 0.06% | 0.26% |
| 3-months (↓) | 0.06% | 0.24% |

The results of this example demonstrate that when a composition according to the invention is stored at up to 40° C. for up to three months, the total concentration of impurities in the composition is at most 0.26%.

All references, including publications, patent applications, and patents, cited herein are hereby incorporated by reference to the same extent as if each reference was individually and specifically indicated to be incorporated by reference and was set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it was individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Other than in the operating examples, or where otherwise indicated, all numbers expressing quantities of ingredients, reaction conditions, and so forth used in the specification and claims are to be understood as being modified by the term “about.” Accordingly, unless indicated to the contrary, the numerical parameters set forth herein are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in a given testing measurement.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition, comprising:
voriconazole,
hydroxypropyl β-cyclodextrin (HPCD), and
an excipient selected from the group consisting of an amino acid and a disaccharide;
where the composition is a solid.

2. The composition of claim 1, where the molar ratio of voriconazole to HPCD is from 1:2 to 1:5.5.

3. The composition of claim 1, where the molar ratio of voriconazole to HPCD is from 1:2.5 to 1:4.

4. The composition of claim 1, where the molar ratio of voriconazole to the excipient is from 1:2.5 to 1:20.

5. The composition of claim 1, where the molar ratio of voriconazole to the excipient is from 1:5 to 1:15.

6. The composition of claim 1, where the excipient is an amino acid and the amino acid is selected from the group consisting of arginine, lysine and threonine.

7. The composition of claim 1, where the excipient is a disaccharide and the disaccharide is selected from the group consisting of lactose and trehalose.

8. The composition of claim 1, comprising a pH modifier in an amount sufficient to provide a pH in the range of 5.2 to 6.9 when the composition is reconstituted in water.

9. The composition of claim 1, where when the composition is stored at 40° C. for 4 weeks, the total concentration of impurities in the composition is at most 0.65%.

10. The composition of claim 1, where when the composition is stored at 55° C. for 2 weeks, the total concentration of impurities in the composition is at most 2.2%.

11. A composition, comprising:
voriconazole,
hydroxypropyl β-cyclodextrin (HPCD), and
arginine;
where the molar ratio of voriconazole to HPCD is from 1:2.7 to 1:3.5,
the molar ratio of voriconazole to arginine is from 1:9 to 1:11, and
the composition is a solid.

12. The composition of claim 11, comprising a pH modifier in an amount sufficient to provide a pH in the range of 5.5 to 6.7 when the composition is reconstituted in water.

13. The composition of claim 11, where
when the composition is stored at 40° C. for 4 weeks, the total concentration of impurities in the composition is at most 0.25%, and
when the composition is stored at 55° C. for 2 weeks, the total concentration of impurities in the composition is at most 0.5%.

14. A composition, formed by a method comprising:
forming a liquid mixture comprising
a solvent,
voriconazole,
hydroxypropyl β-cyclodextrin (HPCD), and
an excipient selected from the group consisting of an amino acid and a disaccharide; and
lyophilizing the liquid mixture to form a solid composition.

15. The composition of claim 14, where the liquid mixture comprises from 50 to 500 mg voriconazole,
a molar ratio of voriconazole to HPCD of from 1:2 to 1:5.5, and
a molar ratio of voriconazole to the excipient of from 1:2.5 to 1:20.

16. The composition of claim 14, where the liquid mixture comprises from 150 to 250 mg voriconazole,
a molar ratio of voriconazole to HPCD of from 1:2.5 to 1:4, and
a molar ratio of voriconazole to the excipient of from 1:5 to 1:15.

17. The composition of claim 14, where the excipient is an amino acid and the amino acid is selected from the group consisting of arginine, lysine and threonine.

18. The composition of claim 14, where the excipient is a disaccharide and the disaccharide is selected from the group consisting of lactose and trehalose.

19. The composition of claim 14, where the liquid mixture comprises a pH modifier in an amount sufficient to provide a pH of from 5.5 to 6.7 prior to the lyophilizing.

20. The composition of claim 14, where
when the composition is stored at 40° C. for 4 weeks, the total concentration of impurities in the composition is at most 0.65%, and
when the composition is stored at 55° C. for 2 weeks, the total concentration of impurities in the composition is at most 2.2%.
